(19) United States
(12) Patent Application Publication
(10) Pub. No.: US 2009/0270298 A1
(43) Pub. Date: Oct. 29, 2009

(54) COSMETIC COMPOSITION WITH A HEATING EFFECT

(75) Inventor: Delphine COMPAIN, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/429,225

(22) Filed: Apr. 24, 2009

Related U.S. Application Data
(60) Provisional application No. 61/050,633, filed on May 6, 2008.

(30) Foreign Application Priority Data
Apr. 25, 2008 (FR) 08 52805

(51) Int. Cl. A61K 8/18; A61K 8/49; A61Q 19/00; A61Q 19/10; A61Q 1/14; A61K 8/58

(52) U.S. Cl. 510/136; 510/130

(57) ABSTRACT

Cosmetic composition containing, in an anhydrous medium, at least one exothermic metal salt, at least one carboxylic acid and at least one salicylic acid compound, chosen from salicylic acid and salicylic acid derivatives. The composition may be used in particular for cleansing and/or removing makeup from and/or scrubbing the skin.

### REFERENCE TO PRIOR APPLICATIONS

This application claims priority to U.S. provisional application Ser. No. 61/050,633, filed May 6, 2008; and to French patent application 08 52805, filed April 25, 2008, both incorporated herein by reference.

### FIELD OF THE INVENTION

The present invention relates to an anhydrous composition comprising a salicylic acid compound, a metal salt and a carboxylic acid, and the uses of the composition in, for example, the cosmetic field, in particular for cleansing and/or removing makeup from and/or scrubbing the skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is known to use anhydrous cosmetic compositions that are exothermic, that is to say that have a heating effect during application to the skin. The heating products may contain various raw materials, such as for example exothermic metal salts which, in the presence of water, release heat.

These heating compositions may comprise care active agents such as for example salicylic acid or its derivatives. However, the simultaneous presence of exothermic metal salts and salicylic acid in these compositions may be accompanied by the appearance of red dots which may appear between 24 hours and 1 month after the manufacture of the product, the reaction being further accelerated by heat. These red dots are unaesthetic and damage the stability of the product, which is unacceptable for the user.

### SUMMARY OF THE INVENTION

The inventor has discovered that the introduction of an acid into a composition comprising an exothermic metal salt and a salicylic acid compound makes it possible to avoid these disadvantages.

### DETAILED DESCRIPTION OF THE PREFERRED EMBODIMENTS

As used herein, the words “a” and “an” and the like carry the meaning of “one or more.”

One subject of the invention is a composition comprising, in an anhydrous medium, an exothermic metal salt, a salicylic acid compound, and an acid.

Preferably, the invention compositions are stable and do not have red dots, even after 3 months at room temperature or after placing in an oven at 50° C., and have a satisfactory hot effect.

The preferred compositions of the invention are exothermic, that is to say that they have a heating effect and therefore that the user feels heating during the application of the composition to wet or moistened skin. Preferably, the invention compositions are those whose temperature in the presence of water (the water added during use or alternatively the water present in the skin) can increase by several degrees (one to twenty degrees) essentially instantly, or instantly.

The composition according to the invention preferably uses an anhydrous medium. In a preferred embodiment the entire composition is anhydrous. The expression “anhydrous” is understood here to mean a practically anhydrous medium, that is to say generally comprising less than 6% by weight of water, preferably less than 4% by weight of water, and better still less than 1% by weight of water relative to the total weight of the composition. Both the medium and the entire composition are each preferably completely anhydrous and therefore free of water.

When the composition of the invention is a cosmetic composition and therefore intended for topical application, it preferably comprises a physiologically acceptable medium, that is to say compatible with the skin, the mucous membranes and/or keratin fibres.

The composition of the invention is preferably homogenous and stable over time. It is generally provided in the form of a translucent to opaque gel, but it may also be provided in any other form, including that of a cream, a paste or even a powder if all the constituents used are powdery, it being possible for composition, whatever its form, to be used as it is or in a sponge or alternatively impregnated into a support such as a wipe, a pad, a dry film, a patch.

Salicylic Acid Compound

The salicylic acid compound present in the composition according to the invention is chosen from salicylic acid and the compounds of the following formula (I):

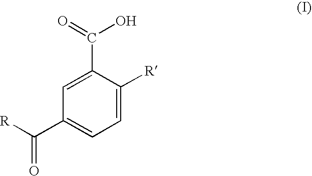

in which:
the radical R denotes a saturated, linear, branched or cyclic aliphatic chain having from 2 to 22 carbon atoms; an unsaturated chain having from 2 to 22 carbon atoms containing one or more double bonds which may be conjugated; an aromatic ring attached to the carbonyl radical directly or via saturated or unsaturated aliphatic chains having from 2 to 7 carbon atoms, it being possible for the groups to be substituted with one or more substituents, which are identical or different, chosen from (a) halogen atoms, (b) a trifluoromethyl group, (c) hydroxyl groups in free form or in a form esterified with an acid having from 1 to 6 carbon atoms or (d) a carboxyl functional group in free form or in a form esterified with a lower alcohol having from 1 to 6 carbon atoms;
R′ is a hydroxyl group;
and their salts derived from an inorganic or organic base.

It is also possible to use a mixture of several such salicylic acid compounds.

Preferably, the radical R denotes a saturated, linear, branched or cyclic aliphatic chain containing from 3 to 11 carbon atoms; an unsaturated chain containing from 3 to 17 carbon atoms and comprising one or more conjugated or unconjugated double bonds; it being possible for the hydrocarbon chains to be substituted with one or more substituents, which are identical or different, chosen from (a) halogen atoms, (b) a trifluoromethyl group, (c) hydroxyl groups in free form or in a form esterified with an acid having from 1 to 6 carbon atoms or (d) a carboxyl functional group in free form or in a form esterified with a lower alcohol having from 1 to 6 carbon atoms.

The compounds more particularly preferred are those in which the radical R is a C₃-C₁₁ alkyl group.

Among the compounds of formula (I) which are particularly preferred, there may be mentioned: 5-(n-octanoyl)salicylic acid (or capryloylsalicylic acid); 5-(n-decanoyl)salicylic acid; 5-(n-dodecanoyl)-salicylic acid; 5-(n-heptyloxy)salicylic acid and their corresponding salts.

The salicylic acid compound is advantageously chosen from salicylic acid and 5-(n-octanoyl)salicylic acid. Use will be more particularly made of 5-(n-octanoyl)salicylic acid.

The salts of the compounds of formula (I) may be obtained by salification with an inorganic or organic base. By way of example of an inorganic base, there may be mentioned alkali or alkaline-earth metal hydroxides such as sodium hydroxide, potassium hydroxide or aqueous ammonia.

Among the organic bases, there may be mentioned amines and alkanolamines. Quaternary salts such as those described in patent FR 2 607 498 are particularly advantageous.

The compounds of formula (I) which can be used according to the invention are described in patents U.S. Pat. No. 6,159,479 and U.S. Pat. No. 5,558,871, FR 2,581,542, FR 2 607 498, U.S. Pat. No. 4,767,750, EP 378,936, U.S. Pat. No. 5,267,407, U.S. Pat. No. 5,667,789, U.S. Pat. No. 5,580,549, EP-A-570 230.

The salicylic acid compound as described above may be present in the emulsion according to the invention in any amount including, for example, from 0.01% to 10% by weight, relative to the total weight of the composition, preferably ranging from 0.05% to 5% by weight, and more preferably ranging from 0.05% to 2% by weight.

Exothermic Metal Salts

The metal salts used in the present invention are salts having exothermic properties, that is to say salts which release heat when they are moistened. They are chosen in particular from alkaline-earth metal salts and magnesium salts.

As alkaline-earth metal salts, there may be mentioned in particular calcium salts and, more especially, calcium halides such as calcium iodide, chloride and bromide. Calcium chloride is preferably used.

As magnesium salts, magnesium sulphate may be mentioned in particular.

It is also possible to use a mixture of several such salts.

According to a preferred embodiment of the invention, the exothermic metal salt is chosen from magnesium sulphate, calcium chloride and mixtures thereof.

The quantity of metal salt(s) is not limited and may range for example from 0.1 to 50% by weight, preferably from 5 to 40% by weight and even better from 10 to 30% by weight relative to the total weight of the composition.

Carboxylic Acid

The carboxylic acid, a separate and different compound from the salicylic acid compound, may be preferably chosen from acids having a pKa ranging from 2 to 6, preferably from 3 to 5. It may be chosen in particular from lactic acid, citric acid, glycolic acid, ascorbic acid, tartaric acid and mixtures thereof.

Preferably, the carboxylic acid is chosen from citric acid and/or glycolic acid.

The carboxylic acid may be present in any quantity including a quantity ranging from 0.01 to 10% by weight, preferably from 0.1 to 5% by weight and even better from 0.5 to 3% by weight relative to the total weight of the composition.

According to one embodiment, the composition comprises at least one silica chosen from hydrophilic silicas, hydrophobic silicas and mixtures thereof. The quantity of silica(s) may range for example from 0.1 to 20% by weight, preferably from 0.5 to 10% by weight and even better from 1 to 5% by weight relative to the total weight of the composition. The expression “hydrophilic silica” is understood to mean, in the present application, both pure hydrophilic silicas and particles coated with hydrophilic silica.

The hydrophilic silicas which may be used in the composition of the invention are preferably amorphous, and they may be of pyrogenic origin or of precipitated origin. They are generally in pulverulent form.

The pyrogenic silicas are obtained by pyrolysis in a continuous flame at 1000° C. of silicon tetrachloride (SiCl₄) in the presence of hydrogen and oxygen.

The precipitated silicas are obtained by reacting an acid with solutions of alkali metal silicates, preferably of sodium silicate.

According to a preferred embodiment of the invention, the hydrophilic silica is chosen from silicas having a specific surface area of 30 to 500 m²/g, and a mean particle size in numerical terms ranging from 3 to 50 nm. They are more particularly the hydrophilic silicas described in Tables (1) and (2) below, and mixtures thereof.

TABLE (1)

| | Trade name | | | |
|---|---|---|---|---|
| | AEROSIL | AEROSIL | | AEROSIL |
| | 90 | 130 | AEROSIL 150 | 200 |
| | (Company | (Company | (Company | (Company |
| | Degussa- | Degussa- | Degussa- | Degussa- |
| | Hüls) | Hüls) | Hüls) | Hüls) |
| Mode of | pyrogenation | pyrogenation | pyrogenation | pyrogenation |
| production | | | | |
| BET surface | 90 ± 15 | 130 ± 25 | 150 ± 15 | 200 ± 15 |
| area (m²/g) | | | | |
| Mean | 20 | 16 | 14 | 12 |
| particle | | | | |
| size (nm) | | | | |
| Remark | | | | size of the |
| | | | | aggregates: |
| | | | | 10-30 and |
| | | | | 200μ |

TABLE (2)

| | Trade name | | | |
|---|---|---|---|---|
| | AEROSIL | | AEROSIL | |
| | 300 | AEROSIL 380 | OX | SILICE FK |
| | (Company | (Company | 50 | 320 DS |
| | Degussa- | Degussa- | (Company | (Company |
| | Hüls) | Hüls) | Degussa- | Degussa- |
| | | | Hüls) | Hüls) |
| Mode of | pyrogenation | pyrogenation | pyrogenation | precipitation |
| production | | | | |
| BET surface | 300 ± 30 | 380 ± 30 | 50 ± 25 | 170 ± 25 |
| area (m²/g) | | | | |
| Mean | 7 | 7 | 40 | 18 |
| particle | | | | |
| size (nm) | | | | |

The hydrophilic silica that can be used in the composition of the invention may also consist of a particle that is completely or partially coated with silica, in particular an inorganic particle that is completely or partially coated with silica, such as silica beads containing titanium oxide, which are marketed under the name TORAYCERAM S-IT® by the company Toray; silica-alumina microspheres containing titanium oxide (size: 105μ), marketed under the name Z-LIGHT-SPHERE W 1012® by the company Zeelan; particles of amorphous precipitated synthetic silica/titanium oxide (size: 106-500μ), which are marketed under the name NEOSIL PC20S® by the company Crosfield; fibres of nylon-6-silica-titanium oxide (length 2 mm and thickness 2 deniers), marketed under the name FIBERLON Y2® by the company Wackherr; silica coated with titanium dioxide and coated with porous silica (85/5/10) (size: 0.6μ), marketed under the name ACS-0050510® by the company SACI-CFPA; anatase nanotitanium oxide treated with alumina and silica at 40% in water (size: 60 nm, monodisperse) marketed under the name MIRASUN TIW 60® by the company Rhodia Chimie CRA; anatase nanotitanium oxide (60 nm) coated with silica/alumina/cerium IV 15/5/3 in aqueous dispersion at 32%, marketed under the name MIRASUN TIW 160® by the company Rhodia Chimie CRA; anatase nanotitanium oxide treated with alumina and silica (34/4.3/1.7) in aqueous dispersion at 40%, marketed under the name TIOVEIL AQ-N® by the company Uniqema; nanotitanium oxide coated with silica (66/33) (particle size of the titanium dioxide: 30 nm; thickness of silica: 4 nm), marketed under the name MAXLIGHT TS-04® by the company Nichimen Europe PLC; and nanotitanium oxide coated with silica (80/20) (titanium dioxide particle size: 30 nm; thickness of silica: 2 nm) marketed under the name MAXLIGHT TS-042® by the company Nichimen Europe PLC.

Pyrogenic silicas and in particular those marketed under the names AEROSIL (INCI name: silica) and in particular the one marketed under the name AEROSIL 200 by the company Degussa-Hüls are preferably used as hydrophilic silica.

The quantity of hydrophilic silica(s) may range for example from 0.1 to 10% by weight, preferably from 0.5 to 5% by weight and even better from 1 to 3% by weight relative to the total weight of the composition.

The expression “hydrophobic silica” in the present application is understood to mean both pure hydrophobic silicas and particles coated with hydrophobic silica.

The hydrophobic silicas which may be used in the composition of the invention are preferably amorphous and of pyrogenic origin. They are preferably provided in pulverulent form.

The amorphous hydrophobic silicas of pyrogenic origin are obtained from hydrophilic silicas. The latter are obtained by pyrolysis in a continuous flame at 1000° C. of silicon tetrachloride (SiCl₄) in the presence of hydrogen and oxygen. They are then made hydrophobic by a treatment with halogenated silanes, alkoxysilanes or silazanes. The hydrophobic silicas differ from the starting hydrophilic silicas, inter alia, in a lower density of silanol groups and in a smaller adsorption of water vapour.

According to a preferred embodiment of the invention, the hydrophobic silica is chosen from silicas having a specific surface area of 50 to 500 m²/g, and a mean particle size in numerical terms ranging from 3 to 50 nm. They are more particularly the hydrophobic silicas described in Table (3) below, and mixtures thereof.

TABLE (3)

| | Trade name | | | | |
|---|---|---|---|---|---|
| | AEROSIL | AEROSIL | AEROSIL | AEROSIL | AEROSIL |
| | R202 | R805 | R812 | R972 | R974 |
| | (Company | (Company | (Company | (Company | (Company |
| | Degussa- | Degussa- | Degussa- | Degussa- | Degussa- |
| | Hüls) | Hüls) | Hüls) | Hüls) | Hüls) |
| BET | 90 ± 20 | 150 ± 25 | 260 ± 30 | 110 ± 20 | 170 ± 20 |
| surface | | | | | |
| area | | | | | |
| (m²/g) | | | | | |
| Mean | 14 | 12 | 7 | 16 | 12 |
| particle | | | | | |
| size (nm) | | | | | |

The hydrophobic silica used in the composition of the invention may also consist of a particle that is completely or partially coated with silica, in particular an inorganic particle that is completely or partially coated with hydrophobic silica, such as pigments and metal oxides coated with hydrophobic silica. These particles may also have optical properties in the product and on the skin, for example they may have a mattifying or mildly bleaching effect.

As hydrophobic silica, use is preferably made of a hydrophobic pyrogenic silica that has been surface-treated with a dimethylsiloxane, such as that marketed under the name AEROSIL R974 (INCI name: Silica Dimethyl Silylate) by the company Degussa-Hüls.

The quantity of hydrophobic silica(s) may range for example from 0.1 to 10% by weight, preferably from 0.5 to 5% by weight and even better from 1 to 5% by weight relative to the total weight of the composition.

According to a preferred embodiment of the invention, the composition of the invention additionally contains at least one polyol. It is believed, without being bound by theory, that the polyols supplement the exothermic action of the salts.

As polyols, there may be mentioned in particular the polyols having at least 2 hydroxyl groups and at least 3 carbon atoms, such as glycerine, diglycerine, and the glycols such as propylene glycol, dipropylene glycol, butylene glycol, hexylene glycol, polyethylene glycol, and polyethylene glycols having a molecular weight of less than 600, such as PEG-8 (or polyethylene 400), the sugars such as sorbitol, and mixtures thereof.

As polyols, glycerine, butylene glycol, propylene glycol, dipropylene glycol, PEG-8 and mixtures thereof are preferably used.

The quantity of polyol(s) depends of the quantity of salts present in the composition and the heating effect desired. It may range for example from 0.5 to 80% by weight, preferably from 5 to 75% by weight, even better from 10 to 70% by weight and better still from 20 to 65% by weight and better still from 30 to 60% by weight relative to the total weight of the composition.

Anhydrous Medium

The composition according to the invention preferably comprises an oily medium comprising at least one oil. The quantity of oil(s) may range for example from 5 to 80% by weight, preferably from 10 to 60% by weight, even better from 10 to 40% by weight relative to the total weight of the composition.

The expression “oil” is understood to mean a fatty substance that is liquid at room temperature (25° C.).

As oils which can be used in the composition of the invention, there may be mentioned for example:

hydrocarbon oils of plant origin, such as liquid triglycerides of fatty acids comprising from 4 to 10 carbon atoms such as the triglycerides of heptanoic or octanoic acids or alternatively, for example, sweet almond oil, sunflower oil, maize oil, soybean oil, gourd oil, coriander oil, grape seed oil, sesame oil, hazelnut oil, apricot oil (Prunus Armenica oil), macadamia oil, arara oil, oil from runiqeman, avocado oil, the triglycerides of caprylic/capric acids such as those sold by the company Stearineries Dubois or those sold under the names Miglyol 810, 812 and 818 by the company Dynamit Nobel, jojoba oil, shea butter oil;

synthetic esters and ethers, in particular of fatty acids, such as the oils of formulae R¹COOR² and R¹OR² in which R¹ represents the residue of a fatty acid comprising from 8 to 29 carbon atoms, and R² represents a branched or unbranched hydrocarbon chain containing from 3 to 30 carbon atoms, such as for example Purcellin oil or 660084 PCL-LIQUID from the company SYMRISE (mixture of cetylstearyl 2-ethylhexanoate and isopropyl myristate), isononyl isononanoate, isopropyl myristate, isopropyl palmitate, 2-ethylhexyl palmitate (or octyl palmitate), 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, such as propylene glycol dioctanoate, neopentyl glycol diheptanoate and diethylene glycol diisononanoate; and the pentaerythritol esters such as pentaerythrityl tetraisostearate;

linear or branched hydrocarbons, of mineral or synthetic origin, such as volatile or non-volatile paraffin oils, and derivatives thereof, petroleum jelly, polydecenes, hydrogenated polyisobutene such as Parleam® oil;

fatty alcohols having from 8 to 26 carbon atoms, such as cetyl alcohol, stearyl alcohol and a mixture thereof (cetearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluorinated oils such as those described in the document JP-A-2-295912. As fluorinated oils, there may also be mentioned perfluoromethylcyclopentane and perfluoro-1,3-dimethylcyclohexane, sold under the names “FLUTEC PC1®” and “FLUTEC PC3®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “FORALKYL®” by the company Atochem; nonafluoromethoxybutane sold under the name “MSX 4518®” by the company 3M and nonafluoroethoxyisobutane; perfluoromorpholine derivatives, such as 4-trifluoromethyl-perfluoro-morpholine sold under the name “PF 5052®” by the company 3M;

silicone oils such as volatile or non-volatile polymethylsiloxanes (PDMS) containing a linear or cyclic silicone chain, which are liquid or pasty at room temperature, in particular cyclopolydimethyl-siloxanes(cyclomethicones) such as cyclohexasiloxane and cyclopentasiloxane; polydimethylsiloxanes comprising alkyl, alkoxy or phenyl groups which are pendant or at the end of the silicone chain, groups having from 2 to 24 carbon atoms; phenylated silicones such as phenyltrimethicones, phenyldimethicones, phenyltrimethylsiloxydiphenylsiloxanes, diphenyl-dimethicones, diphenylmethyldiphenyl trisiloxanes, 2-phenylethyltrimethylsiloxysilicates, and polymethyl-phenylsiloxanes;

mixtures thereof.

The expression “hydrocarbon oil” is understood to mean, in the list of oils mentioned above, any oil predominantly containing carbon and hydrogen atoms, and optionally ester, ether, fluorinated, carboxylic acid and/or alcohol groups.

When the composition of the invention is used as a makeup-removing composition, it preferably contains at least one makeup-removing oil, to which one or more other oils may be added which may or may not be makeup-removing oils. The makeup-removing oils may be chosen in particular from branched hydrocarbons of mineral origin such as hydrogenated polyisobutene, the fatty acid esters described above, and mixtures thereof. As fatty acid esters which can be used as makeup-removing oils, there may be mentioned more particularly ethylhexyl palmitate, ethylhexyl stearate, isopropyl myristate, isopropyl palmitate, isobutyl palmitate, pentaerythrityl caprate/caprylate, cetearyl isononanoate, isodecyl isononanoate, isononyl isononanoate, isotridecyl isononanoate, 2-ethylhexyl caprate/caprylate, and mixtures thereof.

In addition to the oils indicated above, the composition of the invention may contain other fatty substances, such as fatty acids containing from 8 to 30 carbon atoms, such as stearic acid; silicone resins such as trifluoromethyl-C1-4-alkyldimethicone and trifluoropropyldimethicone; silicone gums (INCI name: Dimethiconol) alone or as a mixture with a silicone oil, such as the product marketed by the company Dow Corning under the name Dow Corning 1501 Fluid, which is a mixture of Dimethiconol and Cyclopentasiloxane in the ratio 14.7/85.3 (dimethiconol); silicone elastomers such as those marketed under the names KSG by the company Shin-Etsu; waxes, for example mineral waxes, waxes of animal origin such as beeswax, waxes of plant origin, hydrogenated oils that are concrete at 25° C., fatty esters and glycerides that are concrete at 25° C., synthetic waxes such as polymethylene wax, silicone waxes; and mixtures thereof.

Additives

The composition may further contain one or more other additives, in particular lipophilic additives, including those which are conventionally used in cosmetic compositions, in particular in cleansing or makeup removing compositions.

There may be mentioned in particular exothermic compounds other than salts and polyols, such as for example zeolites which are silicoaluminates. As zeolites, there may be mentioned in particular activated zeolites, and for example the zeolites A, the zeolites X such as those marketed by the companies Fluka and Union Carbide, the zeolites MAP as described in the document EP-A-384070, the activated zeolites A as described in the document EP-A-187912 and in particular those marketed under the name ADVERA 401N ET ADVERA 402N by the company PQ Corporation.

As additives, mention may also be made of surfactants; perfumes; preservatives; antioxidants; sequestrants; fillers; colorants, cosmetic or dermatological active agents, or mixtures thereof. These adjuvants are used in the usual proportions for cleansing and/or care compositions, and for example from 0.01 to 10% of the total weight of the composition. These adjuvants should be of a nature such that and used in quantities such that they do not disrupt the properties sought for the composition of the invention.

According to a particular embodiment of the invention, the composition according to the invention contains, as fillers, exfoliating particles which will allow scrubbing of the skin. As exfoliating particles, use may be made of the exfoliating or scrubbing particles of mineral, plant or organic origins. Thus, it is possible to use for example polyethylene beads or powder, such as those marketed under the name Microthene MN 727 or Microthene MN 710-20 by the company Equistar or such as the powder marketed under the name Gotalene 120 Incolore 2 by the company Dupont; nylon particles such as those marketed by the company Arkema under the name Orgasol 2002 EXD NAT COS; fibres such as polyamide fibres, such as those marketed by the company Utexbel under the name PULPE POLYAMIDE 12185 TAILLE 0,3 MM, polyvinylchloride powder; pumice stone (INCI name: pumice) such as pumice 3/B from Eyraud; the ground shells of fruit kernels such as the ground products of apricot kernels or of walnut shells; sawdust; glass beads; alumina (aluminium oxide) (INCI name: Alumina) such as the product marketed under the name Dermagrain 900 by the company Marketech International, sugar crystals; beads which melt during application to the skin, such as for example the spheres based on mannitol and cellulose which are marketed under the names Unispheres by the company Induchem, capsules based on agar which are marketed under the names Primasponge by the company Cognis, and the spheres based on jojoba esters which are marketed under the names Floraspheres by the company Floratech; and mixtures thereof.

The composition according to the invention may also contain other fillers such as, for example, talc, modified or unmodified starch, and in particular starches esterified with octenylsuccinic anhydride and more particularly “Aluminium Starch octenyl succinate” such as the product marketed by the company National Starch under the name Dry-Flo.

These fillers (exfoliants and the like) may be present in a quantity ranging for example from 0.5 to 20% by weight, preferably from 1 to 15% by weight, even better from 1 to 10% and better still from 2 to 5% by weight relative to the total weight of the composition.

As active agents, there may be mentioned any care or cleansing active agent normally used in the cosmetic field, and in particular antibacterial agents such as octopirox and triclosan, keratolytic agents, essential oils, vitamins such as vitamin C (ascorbic acid), vitamin A (retinol), vitamin PP (niacinamide), vitamin B3 (panthenol) and their derivatives.

The composition of the invention is particularly suitable for cleansing and/or removing makeup from the skin and/or the mucous membranes and more particularly for cleansing and/or removing makeup from and/or scrubbing the skin.

The subject of the invention is also the cosmetic use of the composition as defined above, for cleansing and/or removing makeup from and/or scrubbing the skin.

The composition of the invention is generally provided in the form of a gel. During use, the composition is applied to the skin (in particular the skin of the face), preferably as a thick layer, and then the hands are moistened and the face is massaged with the wet hands. It is possible to optionally wet the hands again and to again massage the face. The face is finally rinsed. Another method comprises first wetting the skin, and then in applying the composition to the wet skin and massaging the face, after having wetted the hands or otherwise.

Accordingly, a subject of the invention is also a cosmetic method for removing makeup from and/or cleansing and/or scrubbing the skin, consisting in wetting the skin, in applying to the skin the composition as defined above, in massaging and in rinsing the skin.

A subject of the invention is also a cosmetic method for removing makeup from and/or cleansing the skin, comprising applying to the skin the composition as defined above, in wetting the hands, in massaging the face with the wet hands, and in rinsing the skin.

The examples below are given by way of nonlimiting illustration in order to better demonstrate the characteristics of the invention. The quantities are given therein in % by weight.

### EXAMPLE 1

### Cleansing Heating Composition

| | |
|---|---|
| 5-(n-Octanoyl)salicylic acid (Mexoryl | 0.3% |
| SAB from Chimex) | |
| PEG-8 | 53.70% |
| Citric acid | 1% |
| Glycerine | 5% |
| Hydrophilic silica (AEROSIL 200) | 2% |
| Hydrophobic silica (AEROSIL R974) | 1% |
| Cyclopentasiloxane/dimethiconol (Dow | 15% |
| Corning 1501 Fluid) (binder) | |
| Magnesium sulphate and water mixture (98/2) | 20% |

Procedure: PEG-8, citric acid, 5-(n-octanoyl)-salicylic acid and glycerine were mixed in a beaker and the mixture was heated to 75° C. (until complete dissolution of the powder is obtained), and hydrophobic silica and hydrophilic silica were added while mixing until a smooth gel was obtained, followed by magnesium sulphate. The mixture was homogenized with a Raynerie device and then the cyclopentasiloxane/dimethiconol was added.

A whitish gel is obtained which gives a hot effect which is quite perceptible and makes it possible to obtain good cleansing of the skin.

After more than 6 months at room temperature, the composition remains stable and without any red dots.

The composition is divided into 4 samples which are placed for 2 months at 4° C., 25° C., 37° C. and 45° C., respectively: these samples remain stable.

### EXAMPLE 2

### Cleansing Heating Composition

| | |
|---|---|
| 5-(n-Octanoyl)salicylic acid (Mexoryl | 0.3% |
| SAB from Chimex) | |
| PEG-8 | 53.70% |
| Glycolic acid (Glypure 99 from Dupont) | 1% |
| Glycerine | 5% |
| Hydrophilic silica (AEROSIL 200) | 2% |
| Hydrophobic silica (AEROSIL R974) | 1% |
| Cyclopentasiloxane/dimethiconol (Dow | 15% |
| Corning 1501 Fluid) (binder) | |
| Magnesium sulphate and water mixture (98/2) | 20% |
| Alumina (Dermagrain 900) | 2% |

The procedure is similar to that of Example 1.

The product obtained is a whitish gel, which gives a hot effect which is quite perceptible and makes it possible to obtain good cleansing of the skin.

After more than 6 months at room temperature, the composition remains stable and without any red dots.

The composition is divided into 4 samples which are placed for 2 months at 4° C., 25° C., 37° C. and 45° C. respectively: these samples remain stable.

### COMPARATIVE EXAMPLES 3 TO 6

The following compositions have been prepared:

| | Exemple 3 | Exemple 4 | Exemple 5 | Exemple 6 |
|---|---|---|---|---|
| | comparative | comparative | (invention) | (invention) |
| 5-(n-Octanoyl)salicylic | — | 0.3 | 0.3 | 0.3 |
| acid (Mexoryl SAB from | | | | |
| Chimex) | | | | |
| Glycolic acid (Glypure 99 | — | — | — | 1 |
| from Dupont) | | | | |
| Citric acid | — | — | 1 | — |
| Hydrophilic silica | 1 | 1 | 1 | 1 |
| (AEROSIL 200) | | | | |
| Hydrophobic silica | 2 | 2 | 2 | 2 |
| (AEROSIL R974) | | | | |
| Solum Diatomea (60-200 | — | 0.2 | — | — |
| microns) | | | | |
| Jojoba beads (250-600 | — | 0.25 | — | — |
| microns) | | | | |
| (Florabeads | | | | |
| jojoba 28/60 Sunburst | | | | |
| Orange from Floratech) | | | | |
| Polyethylene | 1.5 | — | | |
| powder (Gotalene 120 | | | | |
| Incolore 23 from Dupont) | | | | |
| Polyethylene powder | 2 | — | — | — |
| (Gotalene 120 Bleu 23 from | | | | |
| Dupont) | | | | |
| Cyclopentasiloxane/ | 15 | 15 | 15 | 15 |
| Dimethiconol (Dow Corning | | | | |
| 1501 Fluid) | | | | |
| PEG-8 | qsp 100 | qsp 100 | qsp 100 | qsp 100 |
| PEG-7 | 2 | 2 | 2 | 2 |
| caprylic/capric glycerides | | | | |
| Magnesium sulphate | 20 | 20 | 20 | 20 |
| and water mixture (98/2) | | | | |
| Glycerol | 5 | 5 | 5 | 5 |

| | Exemple 3 | Exemple 4 | Exemple 5 | Exemple 6 |
|---|---|---|---|---|
| Aspect | comparative | comparative | (invention) | (invention) |
| At t = 24 h | no red dots | appearance | no red dots | no red dots |
| after | | of red dots | | |
| preparation | | after 24 h | | |
| | | at room | | |
| | | temperature | | |
| After | no red dots | red dots | no red dots | no red dots |
| several | | | | |
| months | | | | |

The introduction of glycolic or citric acid prevents the appearance of red dots due to the presence of salicylic acid compound in the composition.

The above written description of the invention provides a manner and process of making and using it such that any person skilled in this art is enabled to make and use the same, this enablement being provided in particular for the subject matter of the appended claims, which make up a part of the original description.

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising, in an anhydrous medium, at least one exothermic metal salt, at least one carboxylic acid and at least one salicylic acid compound chosen from salicylic acid and the salicylic acid derivatives of formula (I): 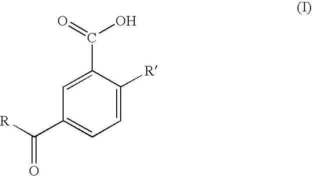
in which:
the radical R denotes a saturated, linear, branched or cyclic aliphatic chain having from 2 to 22 carbon atoms; an unsaturated chain having from 2 to 22 carbon atoms containing one or more double bonds which may be conjugated; an aromatic ring attached to the carbonyl radical directly or via saturated or unsaturated aliphatic chains having from 2 to 7 carbon atoms, it being possible for the groups to be substituted with one or more substituents, which are identical or different, chosen from (a) halogen atoms, (b) a trifluoromethyl group, (c) hydroxyl groups in free form or in a form esterified with an acid having from 1 to 6 carbon atoms or (d) a carboxyl functional group in free form or in a form esterified with a lower alcohol having from 1 to 6 carbon atoms;
R′ is a hydroxyl group; and their salts derived from an inorganic or organic base,
wherein the carboxylic acid is not the salicylic acid compound.

2. The composition according to claim 1, wherein the radical R denotes a saturated, linear, branched or cyclic aliphatic chain containing from 3 to 11 carbon atoms; an unsaturated chain containing from 3 to 17 carbon atoms and comprising one or more conjugated or unconjugated double bonds; it being possible for the hydrocarbon chains to be substituted with one or more substituents, which are identical or different, chosen from (a) halogen atoms, (b) a trifluoromethyl group, (c) hydroxyl groups in free form or in a form esterified with an acid having from 1 to 6 carbon atoms or (d) a carboxyl functional group in free form or in a form esterified with a lower alcohol having from 1 to 6 carbon atoms.

3. The composition according to claim 1, wherein the radical R is a C₃-C₁₁ alkyl group.

4. The composition according to claim 1, wherein the compound of formula (I) is chosen from 5-(n-octanoyl)salicylic acid (or capryloylsalicylic acid); 5-(n-decanoyl)salicylic acid; 5-(n-dodecanoyl)salicylic acid; 5-(n-heptyloxy)salicylic acid and their salts.

5. The composition according to claim 1, wherein the salicylic acid compound is chosen from salicylic acid and 5-(n-octanoyl)salicylic acid.

6. The composition according to claim 1, wherein the salicylic acid compound is present in an amount of 0.05% to 10% by weight, relative to the total weight of the composition.

7. The composition according to claim 1, wherein the exothermic metal salt is chosen from alkaline-earth metal salts and magnesium salts.

8. The composition according to claim 1, wherein the exothermic metal salt is chosen from calcium halides.

9. The composition according to claim 1, wherein the exothermic metal salt is chosen from magnesium sulphate and calcium chloride.

10. The composition according to claim 1, wherein the quantity of exothermic metal salt(s) is 0.1 to 50% by weight relative to the total weight of the composition.

11. The composition according to claim 1, wherein the carboxylic acid is chosen from carboxylic acids having a pKa ranging from 2 to 6.

12. The composition according to claim 1, wherein the carboxylic acid is chosen from lactic acid, citric acid, glycolic acid, ascorbic acid, and tartaric acid.

13. The composition according to claim 1, wherein the carboxylic acid is present in a quantity of 0.01 to 10% by weight relative to the total weight of the composition.

14. The composition according to claim 1, further comprising at least one silica.

15. The composition according to claim 14, wherein the silica is hydrophilic silica.

16. The composition according to claim 14, wherein the silica is hydrophobic silica.

17. The composition according to claim 14, comprising hydrophilic silica and hydrophobic silica.

18. The composition according to claim 14, wherein the quantity of silica(s) is 0.5 to 10% by weight relative to the total weight of the composition.

19. The composition according to claim 17, wherein the compound of formula (I) is chosen from 5-(n-octanoyl)salicylic acid (or capryloylsalicylic acid); 5-(n-decanoyl)salicylic acid; 5-(n-dodecanoyl)salicylic acid; 5-(n-heptyloxy)salicylic acid and their salts, the exothermic metal salt is chosen from alkaline-earth metal salts and magnesium salts, and the carboxylic acid is chosen from lactic acid, citric acid, glycolic acid, ascorbic acid, and tartaric acid.

20. A method for removing makeup from and/or cleansing and/or scrubbing the skin, comprising wetting the skin, applying the composition according to claim 1 to the skin, and massaging and rinsing the skin.
